(19) United States
(12) Patent Application Publication
(10) Pub. No.: US 2019/0365357 A1
(43) Pub. Date: Dec. 5, 2019

(54) SYSTEMS AND METHODS FOR TEMPORAL PERSISTENCE OF DOPPLER SPECTRUM

(71) Applicant: EDAN INSTRUMENTS , INC., Shenzhen (CN)

(72) Inventors: Seshadri SRINIVASAN, Sunnyvale, CA (US); Shangchong YU, Shenzhen (CN); Feng LING, Shenzhen (CN)

(73) Assignee: EDAN INSTRUMENTS, INC., Shenzhen (CN)

(21) Appl. No.: 16/348,143

(22) PCT Filed: Nov. 9, 2016
(86) PCT No.: PCT/CN2016/105169

§ 371 Date: May 7, 2019

(51) Int. Cl. A61B 8/08; A61B 8/06; A61B 8/00

(52) U.S. Cl. CPC A61B 8/5246; A61B 8/06; A61B 8/4427; A61B 8/461; A61B 8/5207; A61B 8/5223; A61B 8/5269; A61B 8/488

(57) ABSTRACT

An ultrasound system (100) includes an ultrasound transducer, a processing circuit (300), and a display. The ultrasound transducer is configured to detect ultrasound information from a patient (610) and output the ultrasound information as an ultrasound data sample (612), the ultrasound information representing blood flow of the patient. The processing circuit (300) is configure to determine a first characteristic of a first plurality of the ultrasound data samples (614) detected prior to a current ultrasound data sample, the first characteristic representing periodic features of the blood flow, determine a second characteristic of the current ultrasound data sample (616), compare the first characteristic to the second characteristic (618), modify the current ultrasound data sample based on the comparison (620), and output spectrum information (622) of the current ultrasound data sample modified based on the comparison of the first characteristic and the second characteristic. The display is configured to display the spectrum information (624).

### TECHNICAL FIELD

The present disclosure generally relates to ultrasound systems. In some implementations, the present disclosure relates to ultrasound systems that can persist historical ultrasound data sample information for displaying ultrasound spectra.

### BACKGROUND ART

Ultrasound systems can be used to detect information regarding a patient, including information regarding blood flow in a patient, in order to display such information to a medical professional or other user so that the user can make medical decisions based on the information. For example, an ultrasound transducer can transmit ultrasound waves into a body of the patient and detect return waves that may have been modified by blood flow and other structural features of the body of the patient, and a computer can communicate with the ultrasound transducer to receive ultrasound information from the ultrasound transducer and display spectra and/or images using the ultrasound information. However, various factors involved in the process of detecting and displaying ultrasound information can reduce the signal to noise ratio of the information ultimately provided to the user, making it difficult to display such information in an accurate and easily understood manner and thus making it difficult for the user to make medical decisions based on the information.

### TECHNICAL PROBLEM

One embodiment relates to a system. The system includes an ultrasound transducer, a processing circuit, and a display. The ultrasound transducer is configured to detect ultrasound information from a patient and output the ultrasound information as an ultrasound data sample, the ultrasound information representing blood flow of the patient. The processing circuit is configure to determine a first characteristic of a first plurality of the ultrasound data samples detected prior to a current ultrasound data sample, the first characteristic representing periodic features of the blood flow, determine a second characteristic of the current ultrasound data sample, compare the first characteristic to the second characteristic, modify the current ultrasound data sample based on the comparison, and output spectrum information of the current ultrasound data sample modified based on the comparison of the first characteristic and the second characteristic. The display is configured to display the spectrum information.

### SOLUTION TO PROBLEM

### Technical Solution

Another embodiment relates to a method. The method includes detecting ultrasound information from a patient by an ultrasound transducer. The method includes outputting the ultrasound information as an ultrasound data sample. The method includes determine a first characteristic of a first plurality of the ultrasound data samples corresponding to ultrasound information detected prior to ultrasound information corresponding to a current ultrasound data sample. The method includes determining a second characteristic of the current ultrasound data sample. The method includes comparing the first characteristic to the second characteristic. The method includes modifying the current ultrasound data sample based on the comparison. The method includes displaying spectrum information based on the modified current ultrasound data sample.

### ADVANTAGEOUS EFFECTS OF INVENTION

### Advantageous Effects

Another embodiment relates to an ultrasound device. The device includes a processing circuit. The processing circuit is configured to receive ultrasound data samples representing velocity of blood flow of a patient, extract a first plurality of traced shapes of a first plurality of the ultrasound data samples detected prior to a current ultrasound data sample, extract a second traced shape of the current ultrasound data sample, compare the first plurality of traced shapes to the second traced shape, modify the current ultrasound data sample based on the comparison; and generate an ultrasound image of a body structure of the patient and the current ultrasound data sample modified based on the comparison of the first plurality of traced shapes and the second traced shape, wherein the modified current ultrasound data sample reduces gaps in a portion of the ultrasound image corresponding to the body structure of the patient.

### BRIEF DESCRIPTION OF DRAWINGS

### Description of Drawings

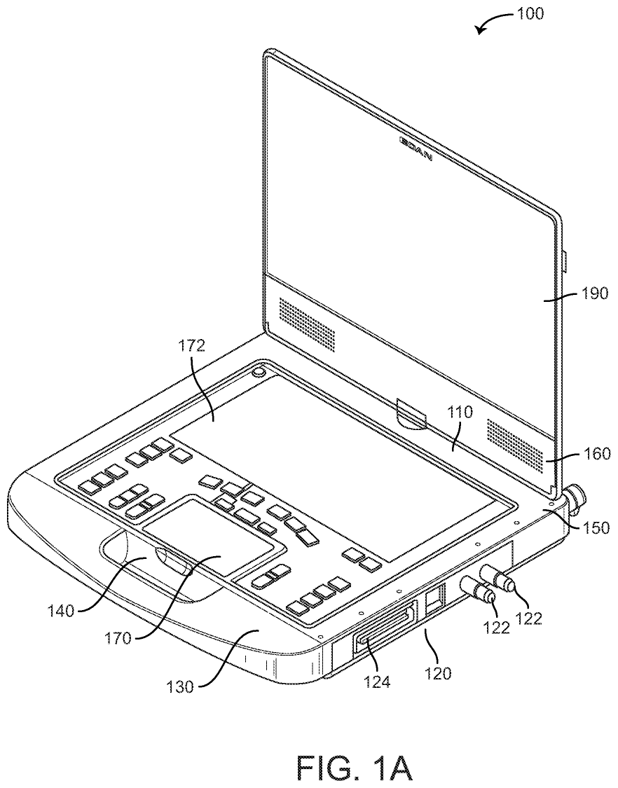

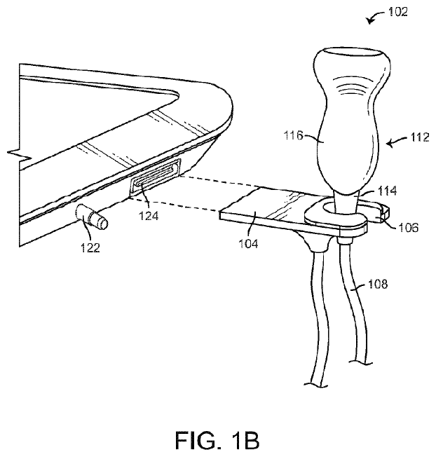

FIG. 1A is a perspective view of an ultrasound system according to an illustrative embodiment.

FIG. 1B is a perspective view of components of an ultrasound system according to an illustrative embodiment.

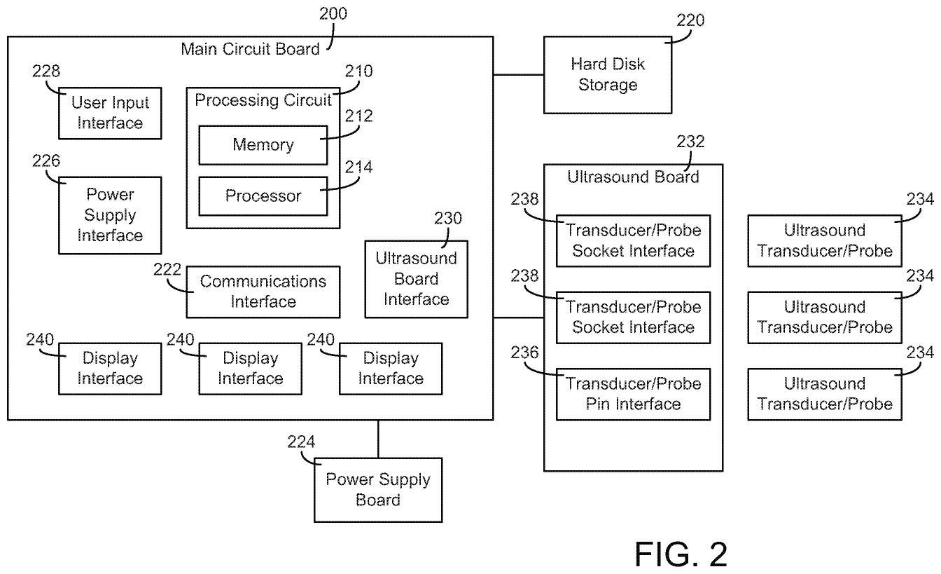

FIG. 2 is a block diagram illustrating components of an ultrasound system according to an illustrative embodiment.

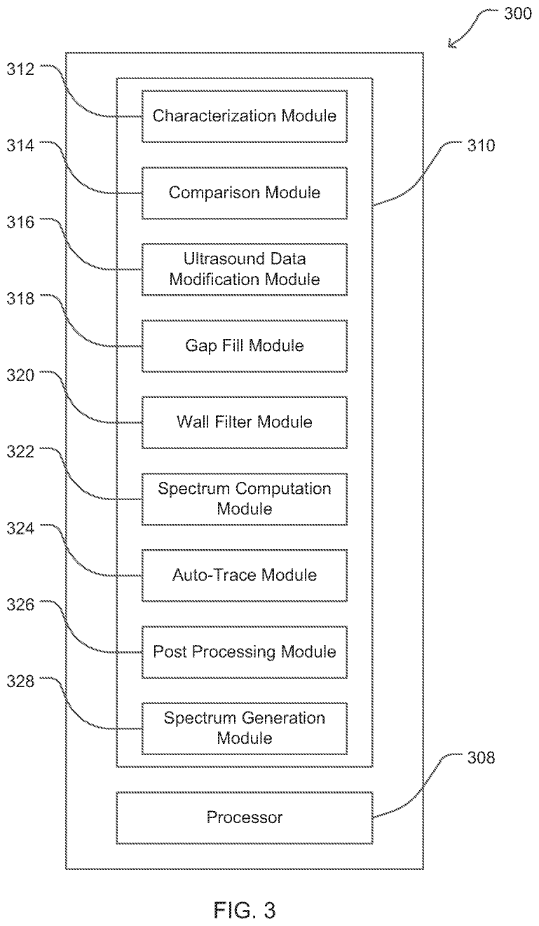

FIG. 3 is a block diagram illustrating components of a processing circuit of an ultrasound system according to an illustrative embodiment.

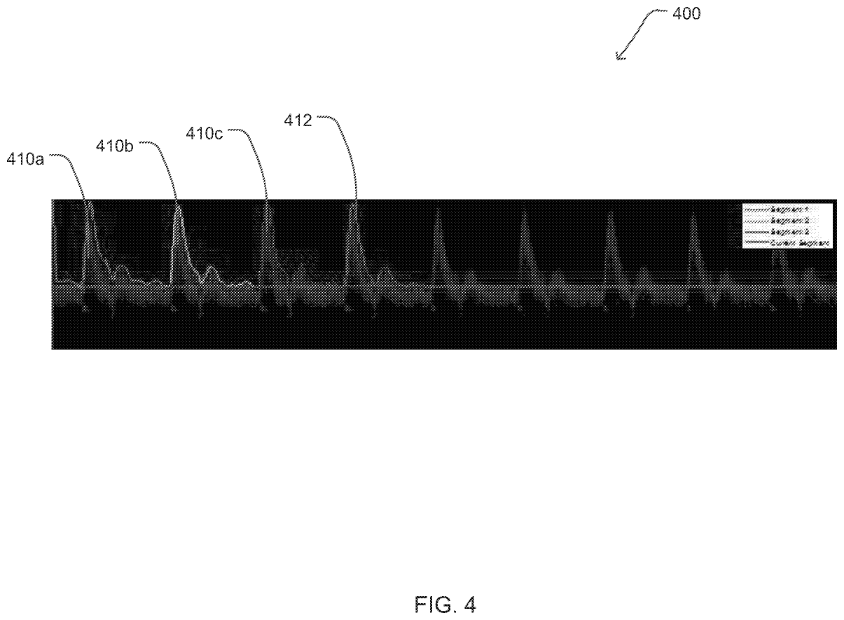

FIG. 4 is a schematic diagram of ultrasound data sample spectra according to an illustrative embodiment.

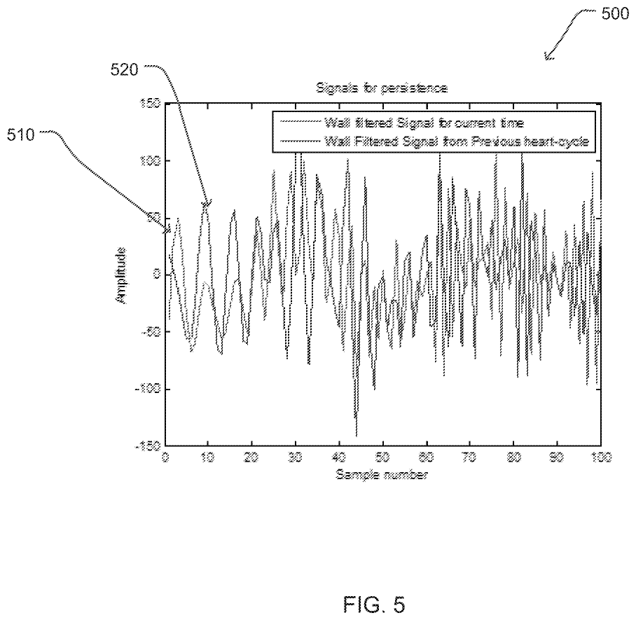

FIG. 5 is a schematic diagram of aligning ultrasound data samples according to an illustrative embodiment.

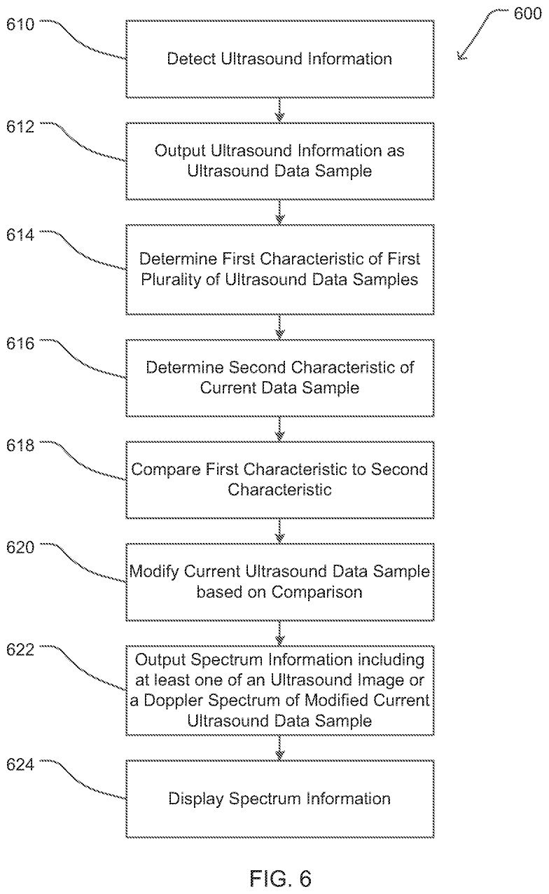

FIG. 6 is a flow chart of a method of modifying a current ultrasound data sample, according an embodiment of the present disclosure.

### MODE FOR THE INVENTION

### Mode for Invention

Before turning to the Figures, which illustrate the exemplary embodiments in detail, it should be understood that the present application is not limited to the details or methodology set forth in the description or illustrated in the figures. It should also be understood that the terminology is for the purpose of description only and should not be regarded as limiting.

Referring to the Figures generally, an ultrasound system can include an ultrasound transducer, a processing circuit, and a display. The ultrasound transducer is configured to detect ultrasound information from a patient and output the ultrasound information as an ultrasound data sample. The ultrasound information represents blood flow of the patient. The processing circuit is configured to determine a first characteristic of a first plurality of the ultrasound data samples detected prior to a current ultrasound data sample. For example, the first plurality of ultrasound data samples can correspond to ultrasound information detected by the ultrasound transducer prior to ultrasound information detected by the ultrasound transducer that is outputted as the current ultrasound data sample. The first characteristic can represent periodic features of the blood flow, such as amplitude information as a function of time of ultrasound signals received by the ultrasound transducer. The processing circuit is configured to determine a second characteristic of the current ultrasound data sample. The processing circuit is configured to compare the first characteristic to the second characteristic. The processing circuit is configured to modify the current ultrasound data sample based on the comparison. For example, depending on how similar the first characteristic and the second characteristic are, the processing circuit can be configured to modify the current ultrasound data sample by including at least a portion of the first plurality of ultrasound data samples, such as by performing a weighted average of (1) the current ultrasound data sample and (2) a composite of the first plurality of ultrasound data samples. The processing circuit is configured to output spectrum information of the current ultrasound data sample modified based on the comparison of the first characteristic and the second characteristic. The spectrum information can include at least one of a Doppler spectrum or an ultrasound image. The display is configured to display the spectrum information. For example, the display can display the spectrum information as a Doppler spectrum; as B-mode or color Doppler images; the display can display the spectrum information in a duplex mode, such as by overlapping ultrasound information representing a structure of the patient's body and blood flow information in and around the structure. The ultrasound system can perform “temporal persistence” by introducing, combining, or otherwise including ultrasound data samples detected prior to a current data sample in an ultrasound spectrum generated based on the current ultrasound data sample.

By modifying the current ultrasound data sample based on the comparison of the first characteristic to the second characteristic, the visualization experience and medical diagnosis operation using the ultrasound system is improved, such as by reducing gaps (or other artefacts) in a duplex mode image display, reducing the significance of noise information that is not common to the first plurality of ultrasound data samples and the current ultrasound data sample, or otherwise improving the ultrasound spectrum. For example, as similar data from the first plurality of ultrasound data samples is propagated into the modified current ultrasound data sample, signal components of the ultrasound data sample can be strengthened, while noise components can be weakened in the displayed ultrasound spectrum.

Referring now to FIG. 1A, one embodiment of portable ultrasound system 100 is illustrated. Portable ultrasound system 100 may include display support system 110 for increasing the durability of the display system. Portable ultrasound system 100 may further include locking lever system 120 for securing ultrasound probes and/or transducers. Some embodiments of portable ultrasound system 100 include ergonomic handle system 130 for increasing portability and usability. Further embodiments include status indicator system 140 which displays, to a user, information relevant to portable ultrasound system 100. Portable ultrasound system 100 may further include features such as an easy to operate and customizable user interface, adjustable feet, a backup battery, modular construction, cooling systems, etc.

Still referring to FIG. 1A, main housing 150 houses components of portable ultrasound system 100. In some embodiments, the components housed within main housing 150 include locking lever system 120, ergonomic handle system 130, and status indicator system 140. Main housing 150 may also be configured to support electronics modules which may be replaced and/or upgraded due to the modular construction of portable ultrasound system 100. In some embodiments, portable ultrasound system 100 includes display housing 160. Display housing 160 may include display support system 110. In some embodiments, portable ultrasound system 100 includes touchpad 170 for receiving user inputs and displaying information, touchscreen 172 for receiving user inputs and displaying information, and main screen 190 for displaying information.

Referring now to FIG. 1B, ultrasound transducer assembly 102 is shown. According to an exemplary embodiment, ultrasound transducer assembly 102 includes a connection assembly to pin (122) or socket (124) type ultrasound interface, shown as ultrasound interface connector 104, coupled to cable 108. Cable 108 may be coupled to a transducer probe 112. While FIG. 1B shows only one transducer assembly 102, more transducer assemblies may be coupled to the ultrasound system 100 based on the quantity of pin (122) or socket (124) type ultrasound interfaces.

Ultrasound interface connector 104 is movable between a removed position with respect to pin (122) or socket (124) type ultrasound interface, in which ultrasound interface connector 104 is not received by pin (122) or socket (124) type ultrasound interface, a partially connected position, in which ultrasound interface connector 104 is partially received by pin (122) or socket (124) type ultrasound interface, and a fully engaged position, in which ultrasound interface connector 104 is fully received by pin (122) or socket (124) type ultrasound interface in a manner that electrically couples transducer probe 112 to ultrasound system 100. In an exemplary embodiment, pin (122) or socket (124) type ultrasound interface may include a sensor or switch that detects the presence of the ultrasound interface connector 104.

In various exemplary embodiments contained herein, the ultrasound interface connector 104 may house passive or active electronic circuits for affecting the performance of the connected transducers. For example, in some embodiments the transducer assembly 102 may include filtering circuitry, processing circuitry, amplifiers, transformers, capacitors, batteries, failsafe circuits, or other electronics which may customize or facilitate the performance of the transducer and/or the overall ultrasound machine. In an exemplary embodiment, ultrasound interface connector 104 may include a bracket 106, where the transducer probe 112 may be stored when not in use.

Transducer probe 112 transmits and receives ultrasound signals that interact with the patient during the diagnostic ultrasound examination. The transducer probe 112 includes a first end 114 and a second end 116. The first end 114 of the transducer probe 112 may be coupled to cable 108. The first end 114 of the transducer probe 112 may vary in shape to properly facilitate the cable 108 and the second end 116. The second end 116 of the transducer probe 112 may vary in shape and size to facilitate the conduction of different types of ultrasound examinations. These first end 114 and second end 116 of transducer probe 112 variations may allow for better examination methods (e.g., contact, position, location, etc.).

A user (e.g., a sonographer, an ultrasound technologist, etc.) may remove a transducer probe 112 from a bracket 106 located on ultrasound interface connector 104, position transducer probe 112, and interact with main screen 190 to conduct the diagnostic ultrasound examination. Conducting the diagnostic ultrasound examination may include pressing transducer probe 112 against the patient's body or placing a variation of transducer probe 112 into the patient. The ultrasound spectrum or image acquired may be viewed on the main screen 190.

Referring to FIG. 2, a block diagram shows internal components of one embodiment of portable ultrasound system 100. Portable ultrasound system 100 includes main circuit board 200. Main circuit board 200 carries out computing tasks to support the functions of portable ultrasound system 100 and provides connection and communication between various components of portable ultrasound system 100. In some embodiments, main circuit board 200 is configured so as to be a replaceable and/or upgradable module.

To perform computational, control, and/or communication tasks, main circuit board 200 includes processing circuit 210. Processing circuit 210 is configured to perform general processing and to perform processing and computational tasks associated with specific functions of portable ultrasound system 100. For example, processing circuit 210 may perform calculations and/or operations related to producing a spectrum and/or an image from signals and/or data provided by ultrasound equipment, running an operating system for portable ultrasound system 100, receiving user inputs, etc. Processing circuit 210 may include memory 212 and processor 214 for use in processing tasks. For example, processing circuit 210 may perform calculations and/or operations.

Processor 214 may be, or may include, one or more microprocessors, application specific integrated circuits (ASICs), circuits containing one or more processing components, a group of distributed processing components, circuitry for supporting a microprocessor, or other hardware configured for processing. Processor 214 is configured to execute computer code. The computer code may be stored in memory 212 to complete and facilitate the activities described herein with respect to portable ultrasound system 100. In other embodiments, the computer code may be retrieved and provided to processor 214 from hard disk storage 220 or communications interface 222 (e.g., the computer code may be provided from a source external to main circuit board 200).

Memory 212 may be any volatile or non-volatile computer-readable storage medium capable of storing data or computer code relating to the activities described herein. For example, memory 212 may include modules which are computer code modules (e.g., executable code, object code, source code, script code, machine code, etc.) configured for execution by processor 214. Memory 212 may include computer code engines or circuits that can be similar to the computer code modules configured for execution by processor 214. Memory 212 may include computer executable code related to functions including ultrasound imagining, battery management, handling user inputs, displaying data, transmitting and receiving data using a wireless communication device, etc. In some embodiments, processing circuit 210 may represent a collection of multiple processing devices (e.g., multiple processors, etc.). In such cases, processor 214 represents the collective processors of the devices and memory 212 represents the collective storage devices of the devices. When executed by processor 214, processing circuit 210 is configured to complete the activities described herein as associated with portable ultrasound system 100, such as for generating ultrasound spectra and/or images (e.g., for display by touchscreen 172 and/or display 190) based on modifying a current ultrasound data sample.

Hard disk storage 220 may be a part of memory 212 and/or used for non-volatile long term storage in portable ultrasound system 100. Hard disk storage 220 may store local files, temporary files, ultrasound spectra and/or images, patient data, an operating system, executable code, and any other data for supporting the activities of portable ultrasound device 100 described herein. In some embodiments, hard disk storage 220 is embedded on main circuit board 200. In other embodiments, hard disk storage 220 is located remote from main circuit board 200 and coupled thereto to allow for the transfer of data, electrical power, and/or control signals. Hard disk storage 220 may be an optical drive, magnetic drive, a solid state hard drive, flash memory, etc.

In some embodiments, main circuit board 200 includes communications interface 222. Communications interface 222 may include connections which enable communication between components of main circuit board 200 and communications hardware. For example, communications interface 222 may provide a connection between main circuit board 200 and a network device (e.g., a network card, a wireless transmitter/receiver, etc.). In further embodiments, communications interface 222 may include additional circuitry to support the functionality of attached communications hardware or to facilitate the transfer of data between communications hardware and main circuit board 200. In other embodiments, communications interface 222 may be a system on a chip (SOC) or other integrated system which allows for transmission of data and reception of data. In such a case, communications interface 222 may be coupled directly to main circuit board 200 as either a removable package or embedded package.

Some embodiments of portable ultrasound system 100 include power supply board 224. Power supply board 224 includes components and circuitry for delivering power to components and devices within and/or attached to portable ultrasound system 100. In some embodiments, power supply board 224 includes components for alternating current and direct current conversion, for transforming voltage, for delivering a steady power supply, etc. These components may include transformers, capacitors, modulators, etc. to perform the above functions. In further embodiments, power supply board 224 includes circuitry for determining the available power of a battery power source. In other embodiments, power supply board 224 may receive information regarding the available power of a battery power source from circuitry located remote from power supply board 224. For example, this circuitry may be included within a battery. In some embodiments, power supply board 224 includes circuitry for switching between power sources. For example, power supply board 224 may draw power from a backup battery while a main battery is switched. In further embodiments, power supply board 224 includes circuitry to operate as an uninterruptable power supply in conjunction with a backup battery. Power supply board 224 also includes a connection to main circuit board 200. This connection may allow power supply board 224 to send and receive information from main circuit board 200. For example, power supply board 224 may send information to main circuit board 200 allowing for the determination of remaining battery power. The connection to main circuit board 200 may also allow main circuit board 200 to send commands to power supply board 224. For example, main circuit board 200 may send a command to power supply board 224 to switch from one source of power to another (e.g., to switch to a backup battery while a main battery is switched). In some embodiments, power supply board 224 is configured to be a module. In such cases, power supply board 224 may be configured so as to be a replaceable and/or upgradable module. In some embodiments, power supply board 224 is or includes a power supply unit. The power supply unit may convert AC power to DC power for use in portable ultrasound system 100. The power supply may perform additional functions such as short circuit protection, overload protection, undervoltage protection, etc. The power supply may conform to ATX specification. In other embodiments, one or more of the above described functions may be carried out by main circuit board 200.

Main circuit board 200 may also include power supply interface 226 which facilitates the above described communication between power supply board 224 and main circuit board 200. Power supply interface 226 may include connections which enable communication between components of main circuit board 200 and power supply board 224. In further embodiments, power supply interface 226 includes additional circuitry to support the functionality of power supply board 224. For example, power supply interface 226 may include circuitry to facilitate the calculation of remaining battery power, manage switching between available power sources, etc. In other embodiments, the above described functions of power supply board 224 may be carried out by power supply interface 226. For example, power supply interface 226 may be a SOC or other integrated system. In such a case, power supply interface 226 may be coupled directly to main circuit board 200 as either a removable package or embedded package.

With continued reference to FIG. 2, some embodiments of main circuit board 200 include user input interface 228. User input interface 228 may include connections which enable communication between components of main circuit board 200 and user input device hardware. For example, user input interface 228 may provide a connection between main circuit board 200 and a capacitive touchscreen, resistive touchscreen, mouse, keyboard, buttons, and/or a controller for the proceeding. In one embodiment, user input interface 228 couples controllers for touchpad 170, touchscreen 172, and main screen 190 to main circuit board 200. In other embodiments, user input interface 228 includes controller circuitry for touchpad 170, touchscreen 172, and main screen 190. In some embodiments, main circuit board 200 includes a plurality of user input interfaces 228. For example, each user input interface 228 may be associated with a single input device (e.g., touchpad 170, touchscreen 172, a keyboard, buttons, etc.).

In further embodiments, user input interface 228 may include additional circuitry to support the functionality of attached user input hardware or to facilitate the transfer of data between user input hardware and main circuit board 200. For example, user input interface 228 may include controller circuitry so as to function as a touchscreen controller. User input interface 228 may also include circuitry for controlling haptic feedback devices associated with user input hardware. In other embodiments, user input interface 228 may be a SOC or other integrated system which allows for receiving user inputs or otherwise controlling user input hardware. In such a case, user input interface 228 may be coupled directly to main circuit board 200 as either a removable package or embedded package.

Main circuit board 200 may also include ultrasound board interface 230 which facilitates communication between ultrasound board 232 and main circuit board 200. Ultrasound board interface 230 may include connections which enable communication between components of main circuit board 200 and ultrasound board 232. In further embodiments, ultrasound board interface 230 includes additional circuitry to support the functionality of ultrasound board 232. For example, ultrasound board interface 230 may include circuitry to facilitate the calculation of parameters used in generating a spectrum and/or an image from ultrasound data provided by ultrasound board 232. In some embodiments, ultrasound board interface 230 is a SOC or other integrated system. In such a case, ultrasound board interface 230 may be coupled directly to main circuit board 200 as either a removable package or embedded package.

In other embodiments, ultrasound board interface 230 includes connections which facilitate use of a modular ultrasound board 232. Ultrasound board 232 may be a module (e.g., ultrasound module) capable of performing functions related to ultrasound imaging (e.g., multiplexing sensor signals from an ultrasound probe/transducer, controlling the frequency of ultrasonic waves produced by an ultrasound probe/transducer, etc.). The connections of ultrasound board interface 230 may facilitate replacement of ultrasound board 232 (e.g., to replace ultrasound board 232 with an upgraded board or a board for a different application). For example, ultrasound board interface 230 may include connections which assist in accurately aligning ultrasound board 232 and/or reducing the likelihood of damage to ultrasound board 232 during removal and/or attachment (e.g., by reducing the force required to connect and/or remove the board, by assisting, with a mechanical advantage, the connection and/or removal of the board, etc.).

In embodiments of portable ultrasound system 100 including ultrasound board 232, ultrasound board 232 includes components and circuitry for supporting ultrasound imaging functions of portable ultrasound system 100. In some embodiments, ultrasound board 232 includes integrated circuits, processors, and memory. Ultrasound board 232 may also include one or more transducer/probe socket interfaces 238. Transducer/probe socket interface 238 enables ultrasound transducer/probe 234 (e.g., a probe with a socket type connector) to interface with ultrasound board 232. For example, transducer/probe socket interface 238 may include circuitry and/or hardware connecting ultrasound transducer/probe 234 to ultrasound board 232 for the transfer of electrical power and/or data. Transducer/probe socket interface 238 may include hardware which locks ultrasound transducer/probe 234 into place (e.g., a slot which accepts a pin on ultrasound transducer/probe 234 when ultrasound transducer/probe 234 is rotated). In some embodiments, ultrasound board 232 includes two transducer/probe socket interfaces 238 to allow the connection of two socket type ultrasound transducers/probes 187.

In some embodiments, ultrasound board 232 also includes one or more transducer/probe pin interfaces 236. Transducer/probe pin interface 236 enables an ultrasound transducer/probe 234 with a pin type connector to interface with ultrasound board 232. Transducer/probe pin interface 236 may include circuitry and/or hardware connecting ultrasound transducer/probe 234 to ultrasound board 232 for the transfer of electrical power and/or data. Transducer/probe pin interface 236 may include hardware which locks ultrasound transducer/probe 234 into place. In some embodiments, ultrasound transducer/probe 234 is locked into place with locking lever system 120. In some embodiments, ultrasound board 232 includes more than one transducer/probe pin interfaces 236 to allow the connection of two or more pin type ultrasound transducers/probes 234. In such cases, portable ultrasound system 100 may include one or more locking lever systems 120. In further embodiments, ultrasound board 232 may include interfaces for additional types of transducer/probe connections.

With continued reference to FIG. 2, some embodiments of main circuit board 200 include display interface 240. Display interface 240 may include connections which enable communication between components of main circuit board 200 and display device hardware. For example, display interface 240 may provide a connection between main circuit board 200 and a liquid crystal display, a plasma display, a cathode ray tube display, a light emitting diode display, and/or a display controller or graphics processing unit for the proceeding or other types of display hardware. In some embodiments, the connection of display hardware to main circuit board 200 by display interface 240 allows a processor or dedicated graphics processing unit on main circuit board 200 to control and/or send data to display hardware. Display interface 240 may be configured to send display data to display device hardware in order to produce a spectrum and/or an image. In some embodiments, main circuit board 200 includes multiple display interfaces 240 for multiple display devices (e.g., three display interfaces 240 connect three displays to main circuit board 200). In other embodiments, one display interface 240 may connect and/or support multiple displays. In one embodiment, three display interfaces 240 couple touchpad 170, touchscreen 172, and main screen 190 to main circuit board 200.

In further embodiments, display interface 240 may include additional circuitry to support the functionality of attached display hardware or to facilitate the transfer of data between display hardware and main circuit board 200. For example, display interface 240 may include controller circuitry, a graphics processing unit, video display controller, etc. In some embodiments, display interface 240 may be a SOC or other integrated system which allows for displaying spectra and/or images with display hardware or otherwise controlling display hardware. Display interface 240 may be coupled directly to main circuit board 200 as either a removable package or embedded package. Processing circuit 210 in conjunction with one or more display interfaces 240 may display spectra and/or images on one or more of touchpad 170, touchscreen 172, and main screen 190.

Referring back to FIG. 1A, in some embodiments, portable ultrasound system 100 includes one or more pin type ultrasound probe interfaces 122. Pin type ultrasound interface 122 may allow an ultrasound probe to connect to an ultrasound board 232 included in ultrasound system 100. For example, an ultrasound probe connected to pin type ultrasound interface 122 may be connected to ultrasound board 232 via transducer/probe pin interface 236. In some embodiments, pin type ultrasound interface 122 allows communication between components of portable ultrasound system 100 and an ultrasound probe. For example, control signals may be provided to the ultrasound probe 112 (e.g., controlling the ultrasound emissions of the probe) and data may be received by ultrasound system 100 from the probe (e.g., imaging data).

In some embodiments, ultrasound system 100 may include locking lever system 120 for securing an ultrasound probe. For example, an ultrasound probe may be secured in pin type ultrasound probe interface 122 by locking lever system 120.

In further embodiments, ultrasound system 100 includes one or more socket type ultrasound probe interfaces 124. Socket type ultrasound probe interfaces 124 may allow a socket type ultrasound probe to connect to an ultrasound board 232 included in ultrasound system 100. For example, an ultrasound probe connected to socket type ultrasound probe interface 124 may be connected to ultrasound board 232 via transducer/probe socket interface 238. In some embodiments, socket type ultrasound probe interface 124 allows communication between components of portable ultrasound system 100 and other components included in or connected with portable ultrasound system 100. For example, control signals may be provided to an ultrasound probe (e.g., controlling the ultrasound emissions of the probe) and data may be received by ultrasound system 100 from the probe (e.g., imaging data).

In various embodiments, various ultrasound imaging systems may be provided with some or all of the features of the portable ultrasound system illustrated in FIGS. 1A-1B and -2. In various embodiments, various ultrasound imaging systems may be provided as a portable ultrasound system, a portable ultrasound transducer, a hand-held ultrasound device, a cart-based ultrasound system, an ultrasound system integrated into other diagnostic systems, etc.

Referring now to FIG. 3, an embodiment of a processing circuit 300 of an ultrasound system (e.g., ultrasound system 100) is illustrated. The processing circuit 300 includes a memory 310 and a processor 312. The processing circuit 300 can be similar to and perform similar functions as the processing circuit 210 described herein with reference to FIG. 2. For example, the memory 310 can be similar to the memory 212, and the processor 312 can be similar to the processor 214. As described herein with reference to FIG. 3, the processing circuit 300 (and particularly, memory 310 thereof) can include various electronic modules (e.g., characterization module 312, etc.), configured to execute various functions performed by an ultrasound system; in various embodiments, the processing circuit 300 can be organized in various ways for determining how functions are executed. The modules can be configured to share responsibilities by sending instructions to each other to execute algorithms and other functions, and receiving outputs generated by the module receiving the instructions.

In some embodiments, the processing circuit 300 is configured to determine characteristics of ultrasound data samples. The processing circuit 300 can receive ultrasound data samples from an ultrasound transducer (e.g., an ultrasound transducer similar or identical to ultrasound transducer assembly 102). The ultrasound data sample can correspond to or represent ultrasound information such as features of blood flow of the patient. The ultrasound data sample can be raw data from the ultrasound transducer. For example, the ultrasound data sample can be an analog radio frequency signal outputted by the ultrasound transducer, or a digital data signal resulting from processing of the analog radio frequency signal by an analog-to-digital converter. The ultrasound data sample can represent a velocity of blood at a single point or within a region in space in the patient.

The ultrasound data sample can correspond to individual points of ultrasound information (e.g., a single point corresponding to amplitude, frequency, time, and/or position information; a single point corresponding to a velocity and time pair), or can be organized into segments corresponding to durations of time, such as durations of time corresponding to a heart cycle of a patient (e.g., sequences of points corresponding amplitude, frequency, time, and/or position information; sequences of points corresponding to velocities paired with times of a heart cycle of a patient). For example, an ultrasound data sample can include a sequence of data point pairs (e.g., raw data) of [frequency, time] corresponding to a heart cycle; or, if a Doppler equation algorithm has been executed to process the raw data, the ultrasound data sample can include a sequence of data point pairs of [velocity, time] corresponding to a heart cycle, or any other sequence of data point pairs corresponding to a Doppler spectrum based on the ultrasound information. In some embodiments, rather than a first plurality of ultrasound data samples, a single first ultrasound data sample is used for possible inclusion when modifying the current ultrasound data sample. This can be beneficial in systems and patient conditions where blood flow is relatively dynamic, by emphasizing only the current and most recent ultrasound data sample while still providing the benefit of persisting signal information from the prior ultrasound data sample.

The processing circuit 300 can include a characterization module 312. The characterization module is configured to determine characteristics of ultrasound data samples. The characterization module 312 can determine a first characteristic for a first plurality of ultrasound data samples detected prior to a current ultrasound data sample, and determine a second characteristic of the current ultrasound data sample. In some embodiments, the characteristic is a representation of an analog signal outputted by the ultrasound transducer assembly 102, such as a magnitude of the analog signal as a function of time. In some embodiments, the characteristic is velocity information as a function of time (e.g., dimensional velocity in units of distance per time at different points in time, velocity normalized to a scale, nondimensional velocity, etc.). If the ultrasound data sample represents a single velocity at a single point in time, the characteristic can be the single velocity. In some embodiments, if the ultrasound data sample represents multiple velocities at multiple points in time (e.g., multiple velocities corresponding to a heart cycle of the patient), the characteristic can be the multiple velocities over time, or the characteristic can represent the multiple velocities (e.g., an average velocity, a weighted average velocity, multiple velocities exceeding or not exceeding a threshold or falling within a range, such a threshold or range related to the heart cycle or other physiological parameter, a number of velocities exceeding or not exceeding the threshold, etc.). For example, by corresponding each ultrasound data sample to a heart cycle, the ultrasound data samples can be compared or otherwise manipulated by aligning the start of each ultrasound data sample with the start of the heart cycle, and thus with each other. In some embodiments, the characteristic is received from the auto-trace module 324 (e.g., the characteristic is an extracted shape of the ultrasound data samples). The characteristic can be determined by integrating the ultrasound data sample (or one or more portions thereof) over time.

In some embodiments, the first plurality of ultrasound data samples prior to the current ultrasound data sample correspond to data and/or heart cycles immediately previous to the current ultrasound data sample. For example, the first plurality of ultrasound data samples can be analog signal information detected immediately previous to the current ultrasound data sample, digital signal information sampled from the analog signal information, and/or velocity points corresponding to data and/or heart cycles immediately previous to the current ultrasound data sample. In some embodiments, the first plurality of ultrasound data samples can be selected from amongst a number of historical ultrasound data samples greater than the number to be selected. For example, if the historical ultrasound data sample information corresponds to twenty heart cycles prior to a heart cycle of the current ultrasound data sample, the second characteristic can be determined based on a subset of the twenty heart cycles (e.g., every other heart cycle, the most recent five heart cycles, five randomly selected heart cycles, a number of heart cycles weighted towards recent cycles but going as far back as the twentieth heart cycle, or any other such combination of heart cycles). If the historical ultrasound data sample information corresponds to ten thousand data values as a function of time, the second characteristic can be determined based on 128 ultrasound data values, such as 128 values spread periodically throughout the ten thousand values; any other such combination of values and values used to determine characteristics can be used. The number of ultrasound data samples used can be determined based on factors such as desired image quality, frame refresh rate, or other user experience factors, computational resources of the processing circuit 300, etc.

In some embodiments, the characteristic represents matching between the ultrasound data sample and a template or expected ultrasound data sample (e.g., a template ultrasound data sample stored in memory 310 or generated based on historical ultrasound data sample information). For example, the characterization module 312 can be configured to perform pattern matching algorithms such as described herein (e.g., sum of absolute differences correlation, cross-correlation, template matching, etc.).

The characterization module 312 can be configured to determine a characteristic for a first plurality of ultrasound data samples. The characterization module 312 can determine a plurality of characteristics corresponding to each ultrasound data sample, and combine the characteristics. For example, the characterization module 312 can determine an average characteristic, such as by averaging extracted shapes of the ultrasound data samples (e.g., by sending an instruction to the auto-trace module 324 to extract a shape for each of the ultrasound data samples, such as a shape having shape information as a function of time, and averaging the shape information for each point in time across the ultrasound data samples). The characterization module 312 can be configured to determine an average characteristic weighted based on a physiological parameter related to a heart cycle of the patient, or a blood flow rate or velocity. For example, if the first plurality of ultrasound data samples correspond to a plurality of heart cycles previous to a heart cycle of a current ultrasound data sample, a characteristic can be determined for each of the first plurality of ultrasound data samples, and then averaged to determine an average characteristic for the first plurality of ultrasound data samples. The characterization module 312 can be configured to determine an average characteristic weighted by recency relative to a time at which a current ultrasound data sample was detected.

In some embodiments, the characterization module 312 is configured to determine a characteristic based on expected features of a heart cycle. The characteristic can be based on expected peaks or troughs of blood velocity during the heart cycle. For example, if a portion of the heart cycle includes an expected peak, the characteristic can be at least partly determined by comparing the velocity of the ultrasound data sample to the velocity of the expected peak. In some embodiments, the heart cycle includes an expected peak and an expected trough, and the characteristic is determined by comparing a first velocity to the expected peak and the second velocity to the expected trough. The characterization module 312 can determine the characteristic before or after execution of the auto-trace module 324.

The processing circuit 300 can include a comparison module 314. The comparison module 314 is configured to compare characteristics of ultrasound data samples, such as to determine similarities (or differences) between ultrasound data samples, or between ultrasound data samples and a template characteristic (e.g., a template characteristic representing expected characteristics, such as expected characteristics determined based on physiological parameters of the patient such as blood flow rate or velocity, expected characteristics determined based on historical characteristic information, etc.). The comparison module 314 can be configured to output an indication of the comparison; for example, if the comparison is a similarity determination, the comparison module 314 can be configured to output a value of “0” if the first characteristic has no similarity to the second characteristic, a value of “1” if the first characteristic is identical to the second characteristic, and a value increasing from 0 to 1 as the similarity increases.

The comparison module 314 can be configured to compare the first characteristic (of the first plurality of ultrasound data samples detected prior to the current ultrasound data sample) to the second characteristic (of the current ultrasound data sample) by measuring a similarity between the first characteristic and the second characteristic. For example, if each characteristic represents a single velocity, the similarity can be a ratio or percentage of the first characteristic relative to the second characteristic. The comparison can be used to determine how much, if any, of the information represented by the first plurality of ultrasound data samples should be used when generating an ultrasound spectrum of the current ultrasound data sample. In general, if the comparison indicates that the first plurality of ultrasound data samples are relatively similar to the current ultrasound data sample, then the comparison may indicate that the first plurality of ultrasound data samples include information representative of a signal component of ultrasound information (e.g., periodic information common to all ultrasound data samples or all heart cycles), rather than a noise component, and the processing circuit 300 can include data of the first plurality of ultrasound data samples when generating an ultrasound spectrum of the current ultrasound data sample, which may improve the clarity and fidelity of the ultrasound spectrum for viewing by a user. The comparison module 314 can execute comparisons of characteristics of raw ultrasound data samples before or after execution of wall-filtering of by the wall filter module 320. The comparison module 314 can execute comparisons of characteristics of ultrasound velocity data or other Doppler spectrum data before or after execution of auto tracing by the auto-trace module 324.

In some embodiments, if each characteristic represents multiple velocities over time (e.g., multiple velocities corresponding to a heart cycle of a patient, or a portion thereof), the similarity can be determined based on comparing each of the velocities of the first characteristic to each of the velocities of the second characteristic. The velocities can be compared by aligning the characteristics in time based on a zero point (e.g., a zero point such as a start of a heart cycle, a midpoint of a heart cycle, a transition point during a heart cycle, and end point of a heart cycle, etc.). The similarity can be determined as an average ratio (or weighted average ratio) of corresponding velocities.

In some embodiments, if each characteristic represents an extracted shape of the ultrasound data sample(s) (e.g., an extracted shape received from the auto-trace module 324), the comparison module 314 can determine the similarity by comparing the extracted shapes. For example, the comparison module 314 can be configured to align each extracted shape in time, and determine ratios or other similarities between corresponding velocities of the extracted shapes. The comparison module 314 can also determine the similarity by comparing the extracted shapes to a template or expected shape.

In some embodiments, the comparison module 314 is configured to compare the first characteristic to the second characteristic based on a sum of absolute differences correlation. The comparison module 314 can determine an absolute difference for each data point of the characteristics, and determine a sum of the absolute differences. For example, if the characteristic is velocity information as a function of time, the comparison module 314 can determine an absolute difference for each velocity data point of each characteristics, and determine a sum of the absolute differences. If the characteristic is a representation of the ultrasound data sample (e.g., an extracted shape as determined by auto-traced module 324), the comparison module can identify components of the characteristic that correspond to one another (e.g., correspond based on periodicity of the ultrasound data sample), and determine the sum of absolute differences correlation based on the corresponding components.

The comparison module 314 can be configured to compare the first characteristic to the second characteristic based on cross-correlation. For example, the comparison module 314 can be configured to execute a sliding dot product algorithm in order to measure similarity of the first characteristic to the second characteristic; as the result of the sliding dot product algorithm increases in magnitude, the similarity will increase, and vice versa.

In some embodiments, the comparison module 314 is configured to compare the first characteristic to the second characteristic based on template matching. For example, the comparison module 314 can include a database storing a template of an ultrasound data sample (or a characteristic thereof), such as a template identifying common features of ultrasound data samples such as velocity information as a function of time. For example, the template can include velocity information as a function of time corresponding to a template or expected heart cycle of the patient. In some embodiments, the template is a template of an auto-traced ultrasound data sample (e.g., an auto-traced ultrasound data sample corresponding to a shape of an ultrasound data sample extracted by auto-trace module 324). In some embodiments, the template is a nondimensional shape of velocity for a heart cycle (e.g., expected velocity magnitudes or amplitudes for each point in time during the heart cycle, normalized to a scale such as a −100 to 100 scale); the template can be multiplied by a physiological parameter such as a flow state parameter (e.g., flow rate, flow velocity, etc.) to dimensionalize the template or otherwise apply the template to the patient and the patient's blood flow.

In some embodiments, the comparison module 314 can be configured to learn the template. For example, the comparison module 314 can store ultrasound data samples received over time from the ultrasound transducer assembly 102, identify common features of the ultrasound data samples (e.g., common velocities or ranges of velocities at points along a heart cycle), and generate the template based on the identified common features.

The processing circuit 300 can include an ultrasound data modification module 316. The ultrasound data modification module 316 can receive an output from the comparison module 314 representing the comparison of the first characteristic to the second characteristic, and generate the ultrasound spectrum based on the comparison. In general, if the comparison indicates that the first characteristic and the second characteristic are relatively similar, the ultrasound data modification module 316 can include more data from the first plurality of ultrasound data samples when modifying the current ultrasound data sample. The data from the first plurality of ultrasound data samples that is used can be normalized to be similar in magnitude to the current ultrasound data sample, to facilitate combining the current ultrasound data sample with the first plurality of ultrasound data samples. For example, if one current ultrasound data sample is to be combined with four ultrasound data samples detected prior to the current ultrasound data sample, the four ultrasound data samples can be averaged (e.g., summed together and then divided by four) or otherwise combined into a single data sample using the methods disclosed herein. In some embodiments, if the output from the comparison module 314 indicates that the first characteristic and the second characteristic are identical (e.g., if the output from the comparison module 314 is “1”), then the modified ultrasound data sample may be identical or nearly identical to each of the current ultrasound data sample and the first plurality of ultrasound data samples, depending on how closely the first characteristic and the second characteristic match the entire features of the respective ultrasound data sample(s). The ultrasound data modification module 316 can be configured to execute data modification before or after execution of wall filtering by the wall filter module 320. The ultrasound data modification module 316 can be configured to execute data modification before or after execution of auto tracing by the auto trace module 324.

The ultrasound data modification module 316 is configured to output a modified ultrasound data sample that includes the current ultrasound data sample. Depending on the output from the comparison module 314, the output may include at least a portion of the first plurality of ultrasound data samples, such as for the spectrum generation module 328 to generate a spectrum of an ultrasound data sample based on the modified ultrasound data sample.

In some embodiments, the ultrasound data modification module 316 modifies the current ultrasound data sample as a weighted average of the current ultrasound data sample and the first plurality of ultrasound data samples (e.g., an average weighted by the output of the comparison module 314). For example, if the output of the comparison module 314 is “0.5,” the current ultrasound data sample can be modifying by weighing (1) the current ultrasound data sample and (2) the first plurality of ultrasound data samples equally. As such, the ultrasound data modification module 316 can be configured to modify the current ultrasound data sample linearly as a function of the output from the comparison module 316.

In some embodiments, the ultrasound data modification module 316 is configured to modify the current ultrasound data sample based on whether the similarity exceeds one or more threshold values. For example, the ultrasound data modification module 316 can be configured to compare the similarity to a first threshold value, and modify the current ultrasound data sample to include at least a portion of the first plurality of ultrasound data samples if the similarity exceeds the first threshold value. The ultrasound data module can be configured to compare the similarity to a second threshold value that is greater than the first threshold value, and modify the current ultrasound data sample to include a greater portion of the first plurality of ultrasound data samples if the similarity exceeds the second threshold value. For example, if the similarity is less than or equal to the first threshold value, no data from the first plurality of ultrasound data samples is included; if the similarity is greater than the first threshold value and less than or equal to the second threshold value, a first portion (e.g., a relatively low portion) of data from the first plurality of ultrasound data samples is included; if the similarity is greater than the second threshold value, a second portion (e.g., a relatively high portion greater than the first portion) of data from the first plurality of ultrasound data samples is included. For example, the ultrasound data modification module 316 can be configured to modify the current ultrasound data sample as output=(1−alpha)*(representation of first plurality of ultrasound data samples)+alpha*(current ultrasound data sample). Alpha can be a proportionality constant determined as (alpha=0 if similarity is less than the first threshold; alpha=alpha_{low} if similarity is greater than or equal to the first threshold and less than the second threshold; alpha=alpha_{high} if similarity is greater than the second threshold). In some embodiments, the characterization module 312 is configured to determine a plurality of first characteristics corresponding to each of the first plurality of ultrasound data samples, the comparison module 314 is configured to determine a plurality of comparisons by comparing each of the plurality of first characteristics to the second characteristic, and the ultrasound data modification module 316 is configured to use a plurality of thresholds (or sets of thresholds) for each of the plurality of comparisons.

The thresholds can be determined based on physiological parameters of a generic patient or of the patient being examined. For example, the thresholds can be a function of a flow state (e.g., a velocity or flow rate of blood flow in the patient). The thresholds can be increased, thus reducing the amount of data from the first plurality of ultrasound data samples included when modifying the current ultrasound data sample, if the flow state indicates high flow rate or high blood flow; similarly, the thresholds can be increased if there is a large change in flow state between the first plurality of ultrasound data samples and the current ultrasound data sample (e.g., if the change in flow state indicates that blood flow properties of the patient have changed significantly, making the historical information represented by the first plurality of ultrasound data samples less relevant). In some embodiments, the thresholds are determined based on user input (e.g., user input received at user interfaces as described herein with reference to FIG. 2).

In some embodiments, the thresholds are increased as a difference in time increases between the time at which the current ultrasound data sample was detected and times at which the first plurality of ultrasound data samples were detected prior to the current ultrasound data samples. For example, if the first plurality of ultrasound data samples are relatively far in time from the current ultrasound data sample, then the first plurality of ultrasound data samples may be less relevant for accurately determining how to display the current ultrasound data sample.

The ultrasound data modification module 316 can be configured to modify the current ultrasound data sample to include a portion of the first plurality of ultrasound data samples based on a nonlinear function. For example, the ultrasound data modification module 316 can use an exponential function, a power law function, or any other nonlinear function to determine the proportion of the first plurality of ultrasound data samples to be included when modifying the current ultrasound data sample. In some embodiments, the ultrasound data modification module 316 is configured to modify the current ultrasound data sample by nonlinearly increasing a portion of the first plurality of ultrasound data samples combined with the current ultrasound data sample as the similarity increases. In various embodiments, various functions and thresholds can be combined. For example, no data from the first plurality of ultrasound data samples could be included if the similarity is less than a first threshold; the amount of data from the first plurality of ultrasound data samples used could increase linearly as the similarity increases from the first threshold to the second threshold; the amount of data from the first plurality of ultrasound data samples used could increase exponentially as the similarity increases from the second threshold to a maximum value (e.g., a maximum value indicating that the first characteristic and the second characteristic are identical).

In some embodiments, the ultrasound data modification module 316 is configured to modify the current ultrasound data sample based on a flow state, such as at least one of a velocity or a flow rate of fluid flow in the patient. The ultrasound transducer assembly 102 can be configured to detect at least one of the velocity or flow rate, and the ultrasound data modification module 316 can receive the at least one of the velocity or flow rate. The processing circuit 300 can be configured to determine the at least one of the velocity or flow rate based on ultrasound information (e.g., ultrasound data samples) received from the ultrasound transducer assembly 102. For example, the velocity can be determined by executing a Doppler equation algorithm based on ultrasound frequency information; the flow rate can be determined by executing an algorithm that combines velocities over a region of space corresponding to the region of interest for determining flow rate. In some embodiments, the flow state is a representation of flow strength, such as a representation that is a function of both velocity and flow rate.

The ultrasound data modification module 316 can modify the current ultrasound data sample based on the flow state by altering a ratio of the first plurality of ultrasound data samples included in or combined with the current ultrasound data sample. For example, if the flow velocity is relatively low, or if the flow rate is relatively low, the current ultrasound data sample can be modified to include more of the first plurality of ultrasound data samples; if the flow velocity is relatively high, or if the flow rate is relatively high, the current ultrasound data sample, can be modified to include less of the first plurality of ultrasound data samples. In some embodiments, such a ratio of the current ultrasound data sample to the first plurality of ultrasound data samples can be proportional to a ratio of a flow state of the current ultrasound data sample to a flow state of the first plurality of ultrasound data samples. For example, the modification of the current ultrasound data sample can be dynamically adapted based on the flow state.

In some embodiments, the processing circuit 300 includes a gap fill module 318. The gap fill module 318 can be configured to fill gaps in ultrasound data (e.g., an analog signal or a digital signal generated by sampling the analog signal) received from the ultrasound transducer assembly 102. Gaps in ultrasound data may occur during points in time in which ultrasound data is not acquired, such as due to limitations in the spatial range of ultrasound transducers. The gap fill module 318 can be configured to repeat an ultrasound data sample acquired previous to a point in time at which a gap occurs, or to interpolate ultrasound data samples previous to and/or following a point in time at which a gap occurs, to fill the gap.

The processing circuit 300 can include a wall filter module 320. The wall filter module 320 is configured to filter the ultrasound data samples to remove features corresponding to walls of blood vessels of the patient prior to determining the characteristics of the ultrasound data samples. For example, the wall filter module 320 can be configured to identify and remove low-frequency components in the ultrasound information detected by the ultrasound transducer assembly 102, such as by applying a high pass filter to the ultrasound information. The high pass filter can be calibrated based on stored information regarding typical frequencies detected for blood flow, as compared to typical frequencies detected for blood vessel walls The high pass filter can be calibrated dynamically and/or in response to user input, such as user input indicating feedback from a user describing whether the displayed spectrum of the ultrasound data samples includes information representative of blood vessel walls. The wall filter module 320 can be interchangeable with the gap fill module 318.

In some embodiments, the wall filter module 320 is configured to filter the ultrasound data samples prior to processing by the characterization module 312, the comparison module 314, and the ultrasound data modification module 316. This can improve the spectrum displayed, such as by removing wall components from the ultrasound data samples that could otherwise introduce noise into the characterization analysis by the characterization module 312.

The processing circuit can include a spectrum computation module 322. The spectrum computation module 322 can be configured to generate Doppler spectrum of ultrasound data samples. The spectrum computation module 322 can receive the ultrasound data sample as Doppler frequency shifts detected by the ultrasound transducer assembly 102, and process the Doppler frequency shifts by executing a Doppler equation algorithm to determine velocity information (e.g., determine velocity information in the time domain, determine velocity information as a function of time and/or space, etc.). In some embodiments, the spectrum computation module 322 is configured to process the ultrasound data samples to identify frequency shifts prior to executing a Doppler equation algorithm to determine velocity information. The spectrum computation module 322 can be configured to output velocity information as paired points (e.g., [velocity, time] pairs).

In some embodiments, the processing circuit 300 includes an auto-trace module 324. The auto-trace module 324 can be configured to execute auto-trace algorithms that identify traced features of ultrasound data samples, such as for the characterization module 314 to determine characteristics of the ultrasound data samples based on the identified traced features. For example, the auto-trace module 324 can extract a traced shape corresponding to velocity and/or amplitude of velocity as a function of time of the ultrasound data samples. In some embodiments, tracing an ultrasound data sample includes identifying velocity values in the ultrasound data sample, and interpolating velocities between consecutive velocity values (e.g., linearly interpolating between velocity values). The auto-trace module 324 can compute an envelope of the ultrasound data signal in the received Doppler spectrum. The auto-trace module 324 can be configured to continuously (e.g., automatically) extract traced shapes of velocity profiles of the ultrasound data samples. The auto-trace module 324 can store a template of a velocity profile (or traced shaped) of a heart cycle, or retrieve the template from another module of the memory 310, and group sequences of velocity and time data point pairs into ultrasound data samples corresponding to heart cycles. For example, the template can indicate expected locations of features such as peaks (e.g., points with increases in velocity prior to the point and decreases in velocity after the point), plateaus (e.g., points with relatively little change in velocity), increases in velocity, decreases in velocity, and/or troughs (e.g., points with decreases in velocity prior to the point and increases in velocity after the point) in a heart cycle, and the auto-trace module 324 can be configured to group sequences of velocity and time data point pairs to align with the expected locations of the features.

In some embodiments, the processing circuit 300 includes a post processing module 326. The post processing module 326 can be configured to process ultrasound data samples, such as by executing gain and/or dynamic range modification algorithms, such as for improving the visual quality of ultrasound spectrum information generated and displayed based on the ultrasound data samples. The post processing module 326 can be interchangeable with the auto-trace module 324.

The processing circuit 300 can include an spectrum generation module 328. The spectrum generation module 328 is configured to generate an ultrasound spectrum or image (e.g., spectrum data corresponding to an ultrasound spectrum and/or image data corresponding to an ultrasound image) based on the current ultrasound data sample, and can output the ultrasound spectrum in a format for display (e.g., for display by touchscreen 172, main display 190, etc.). The spectrum generation module 328 can output the ultrasound spectrum via display interface 240. The spectrum generation module 328 can generate an ultrasound spectrum including an array or matrix of pixels, each pixel corresponding to a displayed point on a display. The spectrum generation module 328 can include color and brightness information with each pixel (e.g., color and brightness information corresponding to an ultrasound data sample to be displayed using one or more pixels).

In some embodiments, the spectrum generation module 328 is configured to generate duplex (and/or triplex) spectrum information for display. For example, the spectrum generation module 328 can generate an ultrasound spectrum or image (or multiple ultrasound spectra or images to be displayed adjacent to one another, superimposed or overlaid, or otherwise coordinated) with a first portion corresponding to a structure of the patient's body (e.g., a two-dimensional image of the structure) and a second portion corresponding to the ultrasound data samples (e.g., corresponding to blood flow information). For example, the spectrum generation module 328 can be configured to use the modified current ultrasound data sample output by the ultrasound data modification module 316 to determine colors for displaying blood flow (e.g., using red to indicate blood flow in a first direction, blue to show blood flow in a second direction, and wavelengths within a red wavelength range (e.g., approximately 620-780 nm) or blue wavelength range (e.g., approximately 455-490 nm) to show magnitude of blood flow).

In some embodiments, the spectrum generation module 328 generates improved ultrasound spectra due to the modification of the current ultrasound data sample using the first plurality of ultrasound data samples detected prior to the current ultrasound data sample. For example, if the duplex or triplex image has gaps (e.g., gaps due to interference, signals being blocked by structures in the body of the patient, etc.), the modification of the current ultrasound data sample can reduce or eliminate gaps, such as by interpolating blood flow information within the space and time in which the gaps occur.

In some embodiments, over time, each ultrasound data sample is modified using a plurality of prior ultrasound data samples. In other words, the processing circuit 300 can be configured to receive and/or store original ultrasound data samples that have not been modified, and also store corresponding modified ultrasound data samples for the full history of ultrasound information received. In some embodiments, when selecting the plurality of prior ultrasound data samples for modification of a current ultrasound data sample, the processing circuit 300 can be configured to use the original ultrasound data samples (rather than modified ultrasound data samples). This can be beneficial in systems and patient conditions where signal to noise ratio may already be relatively high, or where physiological parameters of the patient are relatively dynamic, so that the processing circuit 300 does not inadvertently train to only a subset of features that represent signal data or to outdated features of the blood flow. In some embodiments, the processing circuit 300 can be configured to select at least some modified ultrasound data samples for modification of a current ultrasound data sample. This can be beneficial in systems and patient conditions where signal to noise ratio may be relatively low, or where physiological parameters of the patient may be relatively static, so as to avoid modification of a current ultrasound data sample based on noise features of the prior ultrasound data samples.

Referring now to FIG. 4, an embodiment of an ultrasound spectrum 400 displaying blood flow velocity information is shown. The ultrasound spectrum 400 includes a plurality of ultrasound data samples 410a, 410b, 410c corresponding to ultrasound information detected prior to a ultrasound information of a current ultrasound data sample 412. The ultrasound data samples 410a-410c, 412 can indicate velocity and time information of blood flow of the patient. An ultrasound system (e.g., ultrasound system 100, an ultrasound system including processing circuit 300, etc.) can be configured to modify the current ultrasound data sample to include the plurality of ultrasound data samples of the ultrasound data samples 410a, 410b, 410c. For example, depending on the similarity of the ultrasound data samples 410a, 410b, 410c to the current ultrasound data sample, data of the prior plurality of ultrasound data samples can be included when displaying the current ultrasound data samples 412. As such, the signal to noise ratio of the current ultrasound data sample image 412 is improved.

Referring now to FIG. 5, an embodiment of an alignment map 500 comparing a current ultrasound data sample 510 to a previous ultrasound data sample 520 is shown. The ultrasound data samples 510, 520 correspond to approximately one hundred individual ultrasound data sample points, and as illustrated, have been wall filtered (e.g., filtered by wall filter module 320 to remove low frequency components corresponding to blood vessel walls). In some embodiments, the ultrasound data sample points can be selected such that the ultrasound data samples 510, 520 correspond to a heart cycle of the patient. The processing circuit 300 can be configured to align the current ultrasound data sample 510 with the previous ultrasound data sample 520 to facilitate comparison of a first characteristic of the previous ultrasound data sample 510 to a second characteristic of the current ultrasound data sample 520, such as by aligning amplitudes of the ultrasound data samples corresponding to similar points in time during a heart cycle of the patient.

Referring now to FIG. 6, a method 600 of modifying a current data sample is illustrated. The method 600 can be implemented by an ultrasound system, such as ultrasound system 100, an ultrasound system including processing circuit 300, etc. The method 600 can be performed for displaying an ultrasound spectrum or image to a user performing an ultrasound diagnostic procedure.

At 610, ultrasound information is detected. For example, an ultrasound transducer probe can be positioned adjacent to the patient to detect ultrasound information from the patient.

At 612, the ultrasound information is outputted as an ultrasound data sample. The ultrasound transducer probe can output the ultrasound information as frequency information. In some embodiments, the ultrasound transducer probe can be configured to process the frequency information into velocity information as a function of time, and output the ultrasound data sample as the velocity information as a function of time.

At 614, a first characteristic of a first plurality of ultrasound data samples is determined. The characteristic can be velocity information as a function of time. The characteristic can correspond to a heart cycle of the patient. In some embodiments, determining the first characteristic includes auto-tracing the plurality of ultrasound data samples to extract a shape of the plurality of ultrasound data samples. For example, an auto-trace algorithm can be executed that extracts a shape of the ultrasound data sample representing velocity information over time. At 616, a second characteristic of the current ultrasound data sample is determined. The second characteristic can be determined in a similar manner as the first characteristic, except that the second characteristic is for a single ultrasound data sample, whereas the first characteristic can be a composite measure of characteristics of each of the first plurality of ultrasound data samples, or a characteristic of an average of the first plurality of ultrasound data samples.

At 618, the first characteristic is compared to the second characteristic. For example, velocity information or other values indicated by the characteristics can be compared (e.g., compared in magnitude, proportioned as a ratio, etc.). In some embodiments, comparing the first characteristic to the second characteristic includes measuring a similarity between the first characteristic and the second characteristic. In some embodiments, comparing the first characteristic to the second characteristic includes at least one of executing a sum of absolute differences algorithm, a cross correlation algorithm, or a template matching algorithm. In some embodiments, comparing the first characteristic to the second characteristic includes comparing the characteristics to a template characteristic (e.g., a template characteristic representing a typical or expected heart cycle).

At 620, the current ultrasound data sample is modified based on the comparison. For example, the current ultrasound data sample can be averaged (including a weighted averaged) with the first plurality of ultrasound data samples. In some embodiments, modifying the current ultrasound data sample includes comparing the similarity to one or more threshold values, and modifying the current ultrasound data sample based on whether the similarity exceeds one or more of the threshold values. In some embodiments, modifying the current ultrasound data sample includes linearly or nonlinearly increasing a proportion of the first ultrasound data samples combined with the current ultrasound data sample as the similarity increases.

At 622, image information including at least one of an ultrasound image (e.g., B-mode image, color Doppler, etc.) or a Doppler spectrum of the modified current ultrasound data sample is outputted. The image information can include pixel information, such as an array or matrix of pixels with color and brightness for each pixel. The pixels can be used to represent ultrasound data samples for display, as well as a structure of a body of the patient for displaying duplex mode.

At 624, spectrum information is displayed. For example, a display of an ultrasound system can receive the spectrum information and display the spectrum information to a user (e.g., a medical professional performing an ultrasound diagnostic procedure, a patient, etc.). In some embodiments, displaying the spectrum information includes displaying ultrasound spectra in duplex mode, and the modification of the current ultrasound data sample displayed reduces gaps in the duplex mode ultrasound images.

In some embodiments, the method 600 includes filtering the ultrasound data samples to remove features corresponding to walls of blood vessels of the patient. For example, a high pass filter can be applied to the ultrasound data samples to remove low frequency components of the ultrasound data samples. The high pass filter can be calibrated based on known or expected frequencies of blood flow as compared to known or expected frequencies of blood vessel walls. In some embodiments, filtering the ultrasound data samples occurs prior to determining characteristics of the ultrasound data samples, which can beneficially remove noise components of the ultrasound data samples prior to characterization, helping to focus the characterization on signal components.

The present disclosure contemplates methods, systems, and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, CD-ROM or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. When information is transferred or provided over a network or another communications connection (either hardwired, wireless, or a combination of hardwired or wireless) to a machine, the machine properly views the connection as a machine-readable medium. Thus, any such connection is properly termed a machine-readable medium. Combinations of the above are also included within the scope of machine-readable media. Machine-executable instructions include, for example, instructions and data which cause a general purpose computer, special purpose computer, or special purpose processing machines to perform a certain function or group of functions.

Although the figures may show a specific order of method steps, the order of the steps may differ from what is depicted. Also two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule based logic and other logic to accomplish the various connection steps, processing steps, comparison steps and decision steps.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A system, comprising:
an ultrasound transducer configured to detect ultrasound information from a patient and output the ultrasound information as an ultrasound data sample, the ultrasound information representing blood flow of the patient;
a processing circuit configured to:
determine a first characteristic of a first plurality of the ultrasound data samples detected prior to a current ultrasound data sample, the first characteristic representing periodic features of the blood flow;
determine a second characteristic of the current ultrasound data sample;
compare the first characteristic to the second characteristic;
modify the current ultrasound data sample based on the comparison; and
output spectrum information of the current ultrasound data sample modified based on the comparison of the first characteristic and the second characteristic; and
a display configured to display the spectrum information.

2. The system of claim 1, wherein each ultrasound data sample corresponds to a cycle of a heart of the patient.

3. The system of claim 1, wherein the processing circuit is further configured to compare the first characteristic to the second characteristic by measuring a similarity between the first characteristic and the second characteristic.

4. The system of claim 3, wherein the processing circuit is further configured to:
determine whether the similarity exceeds a first threshold value;
modify the current ultrasound data sample to include at least a first portion of the first plurality of ultrasound data samples if the similarity exceeds the first threshold value;
determine whether the similarity exceeds a second threshold value; and
modify the current ultrasound data sample to include at least a second portion of the first plurality of ultrasound data samples if the similarity exceeds the second threshold value, the second portion being greater in magnitude than the first portion.

5. The system of claim 3, wherein the processing circuit is further configured to modify the current ultrasound data sample by nonlinearly increasing a proportion of the first plurality of ultrasound data samples combined with the current ultrasound data sample as the similarity increases.

6. The system of claim 1, wherein the processing circuit is further configured to filter the ultrasound data samples to remove features corresponding to walls of blood vessels of the patient prior to determining the characteristics of the ultrasound data samples.

7. The system of claim 1, wherein the ultrasound transducer is configured to detect at least one of a velocity or a flow rate of fluid flow in the patient, and the processing circuit is configured to further modify the current ultrasound data sample based on the at least one of the velocity or flow rate of fluid flow in the patient.

8. The system of claim 1, wherein the processing circuit is configured to determine characteristics of the ultrasound data samples by extracting a traced shape corresponding to amplitude as a function of time of the ultrasound data samples.

9. The system of claim 1, wherein the display is configured to display spectrum information in duplex mode, and the modification of the current ultrasound data sample reduces gaps in the duplex mode.

10. The system of claim 1, wherein the processing circuit is configured to compare the first characteristic to the second characteristic based on at least one of sum of absolute differences correlation, cross-correlation, or template matching.

11. The system of claim 1, wherein the spectrum information includes at least one of a Doppler spectrum or an ultrasound image.

12. A method, comprising:
detecting ultrasound information from a patient by an ultrasound transducer;
outputting the ultrasound information as an ultrasound data sample;
determine a first characteristic of a first plurality of the ultrasound data samples corresponding to ultrasound information detected prior to ultrasound information corresponding to a current ultrasound data sample;
determining a second characteristic of the current ultrasound data sample;
comparing the first characteristic to the second characteristic;
modifying the current ultrasound data sample based on the comparison; and
displaying spectrum information based on the modified current ultrasound data sample.

13. The method of claim 12, wherein comparing the first characteristic to the second characteristic further comprises measuring a similarity between the first characteristic and the second characteristic.

14. The method of claim 13, further comprising:
determining whether the similarity exceeds a first threshold value;
modifying the current ultrasound data sample to include at least a first portion of the first plurality of ultrasound data samples if the similarity exceeds the first threshold value;
determining whether the similarity exceeds a second threshold value; and
modifying the current ultrasound data sample to include at least a second portion of the first plurality of ultrasound data samples if the similarity exceeds the second threshold value, the second portion being greater in magnitude than the first portion.

15. The method of claim 13, further comprising modifying the current ultrasound data sample by nonlinearly increasing a proportion of the first plurality of ultrasound data samples combined with the current ultrasound data sample as the similarity increases.

16. The method of claim 12, further comprising filtering the ultrasound data samples to remove features corresponding to walls of blood vessels of the patient prior to determining the characteristics of the ultrasound data samples.

17. The method of claim 12, further comprising determining characteristics of the ultrasound data samples by extracting a traced shape corresponding to amplitude as a function of time of the ultrasound data samples.

18. The method of claim 12, further comprising displaying the spectrum information in duplex mode, wherein modifying the current ultrasound data sample reduces gaps in the duplex mode.

19. An ultrasound device, comprising a processing circuit configured to:
receive ultrasound data samples representing blood flow of a patient;
extract a first plurality of traced shapes of a first plurality of the ultrasound data samples detected prior to a current ultrasound data sample;
extract a second traced shape of the current ultrasound data sample;
compare the first plurality of traced shapes to the second traced shape; modify the current ultrasound data sample based on the comparison; and
generate an ultrasound image of a body structure of the patient and the current ultrasound data sample modified based on the comparison of the first plurality of traced shapes and the second traced shape, wherein the modified current ultrasound data sample reduces gaps in a portion of the ultrasound image corresponding to the body structure of the patient.

20. The ultrasound device of claim 19, wherein the processing circuit is further configured to:
determine a similarity between the first traced shape and the second traced shape;
determine whether the similarity exceeds a first threshold value;
modify the current ultrasound data sample to include at least a first portion of the first plurality of ultrasound data samples if the similarity exceeds the first threshold value;
determine whether the similarity exceeds a second threshold value; and
modify the current ultrasound data sample to include at least a second portion of the first plurality of ultrasound data samples if the similarity exceeds the second threshold value, the second portion being greater in magnitude than the first portion.

21. The ultrasound device of claim 19, wherein the processing circuit is further configured to filter the ultrasound data samples to remove features corresponding to walls of blood vessels of the patient prior to determining the characteristics of the ultrasound data samples.
